(19) United States
(12) Patent Application Publication
(10) Pub. No.: US 2012/0148689 A1
(43) Pub. Date: Jun. 14, 2012

(54) FILM COMPRISING ACTIVE DRUGS

(76) Inventor: Todd Maibach, Menlo Park, CA (US)

(21) Appl. No.: 13/399,115

(22) Filed: Feb. 17, 2012

Related U.S. Application Data
(63) Continuation of application No. 12/141,707, filed on Jun. 18, 2008, now abandoned.

(51) Int. Cl. A61K 31/4525; A61K 31/439; A61K 31/473; A61K 31/5377; A61K 31/352; A61K 31/4184; A61K 31/5415; A61K 31/46; A61K 31/437; A61K 31/55; A61K 31/5513; A61K 31/19; A61K 31/675; A61K 31/196; A61K 31/53; A61K 31/4015; A61K 31/423; A61K 31/4535; A61K 31/513; A61K 31/404; A61K 31/403; A61K 31/422; A61K 31/554; A61K 36/16; A61K 31/40; A61P 25/06; A61P 25/08; A61P 25/00; A61P 1/08; B29C 39/02; A61K 31/4178

(52) U.S. Cl. 424/752; 514/321; 514/397; 514/299; 514/296; 514/230.8; 514/454; 514/394; 514/224.2; 514/304; 514/292; 514/217; 514/221; 514/557; 514/94; 514/561; 514/242; 514/424; 514/379; 514/326; 514/269; 514/414; 514/411; 514/376; 514/211.13; 514/423; 264/330

(57) ABSTRACT

The present invention is related to the composition and methods of manufacture of orally-dissolvable, edible films as a vehicle for the non-invasive administration of active drugs through the mucosal tissues of the oral cavity. The films include a water soluble film-forming polymer such as pullulan. Methods for producing the films are also disclosed.

### CROSS REFERENCE TO RELATED APPLICATIONS

The present application is a Continuation and claims the benefit, under 35 U.S.C. §120, of U.S. patent application Ser. No. 12/141,707, filed Jun. 18, 2008, and claims the benefit, under 35 U.S.C. §119, of Provisional U.S. application Ser. No. 60/944,942, filed Jun. 19, 2007, which claims priority to application Ser. No. 60/900,328 filed Feb. 9, 2007, the entire contents and substance of which is hereby incorporated by reference.

### FIELD OF THE INVENTION

This invention relates to the administration of nitroglycerin, as well as other active drugs, via consumable, edible films.

### BACKGROUND OF THE INVENTION

Nitroglycerin is a powerful vasodilator used to prevent chest pain (angina pectoris) by relaxing the smooth muscle of blood vessels in the heart, increasing blood flow and oxygen to the heart muscle, and reducing the pumping force the heart must exert to circulate blood through the body. This reduction in the heart's workload relieves the pain of angina pectoris. Nitroglycerin also finds additional utility in controlling blood pressure in perioperative hypertension, or hypertension resulting from intratracheal intubation, anesthesia, skin incision, sternotomy, cardiac bypass, and postsurgical recovery, in addition to producing controlled hypotension during surgery.

Existing methods of administration of nitroglycerin include a nitroglycerin pump-spray, nitroglycerin sublingual tablet, nitroglycerin sustained released tablets, nitroglycerin transdermal patches, nitroglycerin 2% ointment, and an intravenous nitroglycerin drip. However, each of these methods have inherent drawbacks.

Oral administration is probably the most prevalent method of administering nitroglycerin because of its convenience. It is generally non-threatening, painless, and simple to accomplish for most patients. Nevertheless, the oral administration of nitroglycerin suffers from several disadvantages. Specific problems associated with the oral administration of compressed sustained-release nitroglycerin tablets include friability, content uniformity, such as weight and dosage variations, migration of nitroglycerin to other tablets, the storage container and container components and the resulting potency loss.

A further problem with oral administration in pill form is that the rate of absorption of the drug into the bloodstream after swallowing varies from patient to patient. The absorption of the drug is dependent upon the movement of the drug from the stomach to the small and large intestines and the effects of secretions from these organs and on the resulting pH within the stomach and intestines. Anxiety and stress can dramatically reduce these movements and secretions, prevent or reduce the final effects of the drug, and delay onset of the drug's effects. Most significant is the fact that there is normally a substantial delay between the time of oral administration and the time that the therapeutic effect of the drug begins.

An additional disadvantage of oral pill form administration is that many drugs almost immediately experience metabolism or inactivation. The veins from the stomach and the small and large intestines pass directly through the liver. Thus, drugs entering the bloodstream must first pass through the liver before distribution into the general blood circulation. More than sixty percent of most drugs (and essentially one hundred percent of certain drugs) are removed from the patient's bloodstream during this “first pass” through the liver. The result is that oral pill form administration is impractical for many drugs, particularly cardiovascular-acting drugs that are used for rapid onset in critical care situations.

In order to avoid some of the disadvantages of oral administration, injection is frequently used. Injecting nitroglycerin intravenously results in rapid entry of the drug into the patient's bloodstream. In addition, this type of delivery avoids the removal of large quantities of the drug by the patient's liver. As a result, less total drug is usually needed compared to orally distributed to various portions of the patient's body before exposure to the liver. However, most patients, particularly children and geriatric adults, have an aversion to injections. In some patients, this aversion may be so pronounced as to make the use of injections a serious concern. Since intense psychological stress can exacerbate a patient's debilitated condition, it sometimes becomes undesirable to use injections where the patient is seriously ill or suffers from a debilitating condition or injury.

Another method of administration of pharmaceutically active agents, such as nitroglycerin, includes the transdermal patch. In this method of administration, a dose of nitroglycerin is administered by absorption through the dermal layers into the blood stream. However, a serious disadvantage of the transdermal patch method of nitroglycerin administration is the development of a drug tolerance within a twenty-four (24) hour period when patches are worn continuously, subsequently reducing the effectiveness of the medication. Revised labeling approved by FDA recommended a dosing schedule alternating a daily patch-on period of 12 to 14 hours a day with a patch-off period of 10 to 12 hours, making this time consuming and easily forgotten. Moreover, the patch cannot be used on parts of the body with hair, cuts, abrasions, calluses or scars, and may lead to skin irritation where the patch is applied.

Some investigators have suggested that it may be possible to administer medication through the buccal mucosa of the cheek pouch or by sublingual administration. See, U.S. Pat. No. 4,671,953, the entire content of which is incorporated by reference herein. Such administration through the mucosal tissues of the mouth, pharynx, and esophagus of therapeutic drugs possesses a distinct usefulness. Administration of drugs by this route does not expose the drug to the gastric and intestinal digestive juices. In addition, the drugs largely bypass the liver on the first pass through the body, thereby avoiding additional metabolism and/or inactivation of the drug. Generally the drugs which are administered by any of the methods described above have an unpleasant taste. As a result, in order to allow for buccal or sublingual administration through the oral mucosal tissues, it is also necessary to incorporate the drug into some type of pleasant tasting mass, such as a “candy” matrix.

For effective application of the drug, a candy product may contain the drug uniformly distributed throughout in order to ensure uniform levels of medication. Alternatively, for some applications, varying concentrations within known and controlled ranges may be desired to vary the rate of drug administration. Difficulties are encountered in attempting to blend solid drugs in a uniform or otherwise carefully controlled manner. Many drugs are insoluble, or only partially soluble, in one or more of the ingredients of the hard candy base. Thus, the resultant product is often found to be lacking in uniform or controlled distribution of the drug. Moreover, sublingual tablets also experience issues related to inter-tablet migration of nitroglycerin, similar to the sustained-release tablet methodology, which can produce a high degree of weight and dose variation between tablets.

Furthermore, many presently available medicated candy lozenges tend to crumble when placed in the mouth. As a result, uniform release of the drug into the mucosal tissues does not take place. Rather, the crumbled lozenge is mostly chewed, and swallowed, and the drug enters the bloodstream through the stomach and intestines as described above. Thus, it will be appreciated that candy lozenges have very definite limitations for use in the administration of a drug through the oral mucosal tissues. As a result, lozenges have not been used to administer potent, fast-acting drugs, such as drugs that affect the central nervous system, the cardiovascular system, or the renal vascular system.

While the administration of certain drugs through the oral mucosal tissues has shown promise, development of a fully acceptable method for producing a medication in a desirable form and administering the medication has been elusive.

It would be an important advancement in the art of orally administering potent, fast-acting drugs, if suitable methods and compositions provided a precise dosage to a precise effect in every patient. It would be a further advancement in the art to provide methods and compositions for uniformly incorporating drugs (including insoluble drugs) into a soluble matrix without heating the mixture to the point that degradation occurs.

A need, therefore, exists for an improved vehicle for the administration of pharmaceutical agents, such as nitroglycerin, beyond existing preparations.

### SUMMARY OF THE INVENTION

The invention provides a physiologically acceptable edible or consumable film, which is particularly well adapted to rapidly dissolve in the mouth of a patient.

The invention is also directed to a method for producing a supple, non-self-adhering film especially suitable for oral delivery of active drugs. The method comprises mixing at least one film forming agent with an aqueous solution to provide a hydrated polymer gel; casting the hydrated polymer gel on a substrate; and allowing the cast gel to solidify to provide a film. In another embodiment, the active drug is added to one or more of the components of the mixture prior to forming the hydrated polymer gel.

In another embodiment of the present invention, the active drug may comprise one or more anti-emetics. Such anti-emetics include and may be selected from one or more of the group consisting of: ondansetron, granisetron, palonosetron, dronabinol, aprepitant, ramosetron, metopimazine, nabilone, tropisetron, metoclopramide, prochlorperazine, trimethobenzamide, dimenhydrinate, prochlorperazine and dolasetron.

In another embodiment of the present invention, the active drug may comprise one or more 5HT3 antagonists. Such 5HT3 antagonists include and may be selected from one or more of the group consisting of: alosetron, ondansetron, granisetron, palonosetron, ramosetron and tropisetron.

In another embodiment of the present invention, the active drug may comprise one or more selective serotonin reuptake inhibitors. Such selective serotonin reuptake inhibitors include and may be selected from one or more of the group consisting of: fluoxetine, sertraline, paroxetine, fluvoxamine, citalopram, and alaproclate.

In another embodiment of the present invention, the active drug may comprise one or more anti-epileptics. Such anti-epileptics include and may be selected from one or more of the group consisting of: carbamazepine, clonazepam, diazepam, divalproex sodium, fosphenytoin, gabapentin, lamotrigine, levetiracetam, oxcarbazepine, phenytoin, pregabalin, primidone, tiagabine, topiramate, valproate sodium, vigabatrin and zonisamide.

In another embodiment of the present invention, the active drug may comprise one or more anti-migraines. Such anti-migraines include and may be selected from one or more of the group consisting of: almotriptan, dihydroergotamine mesylate, eletriptan, frovatriptan, naratriptan, rizatriptan, sumatriptan and zolmitriptan.

In another embodiment of the present invention, the active drug may comprise one or more dopamine D1 and D2 antagonists. Such dopamine D1 and D2 antagonists include and may be selected from one or more of the group consisting of: amisulpride, bromperidol, cabergoline, domperidone, fenoldopam, haloperidol, metoclopramide, metopimazine, pergolide mesylate, prochlorperazine, quetiapine, ropinirole hydrochloride, sulpiride, tiapride and zotepine.

In another embodiment of the present invention, the active drug may comprise one or more nootropics. Such nootropics include and may be selected from one or more of the group consisting of: almitrine dimesylate & raubasine, cevimeline hydrochloride, codergocrine mesylate, donepezil, galantamine, ginkgo biloba extract (EGb 761), memantine, nicergoline, piracetam, rivastigmine, sulbutiamine, tacrine and vinpocetine.

In another embodiment of the present invention, the active drug may comprise one or more statins. Such statins include and may be selected from one or more of the group consisting of: atorvastatin, cerivastatin, fluvastatin, lovastatin, pitavastatin, pravastatin, rosuvastatin and simvastatin.

In another embodiment of the invention, the film may comprise one or more of the anti-emetics, 5HT3 antagonists, selective serotonin reuptake inhibitors, anti-epileptics, anti-migraines, dopamine D1 and D2 antagonists, nootropics and statins listed above and including others known in the art.

In another embodiment of the present invention, one or more active drugs from the consumable film may be excluded. The excluded active drug may be an anti-emetic selected from the group consisting of ondansetron, granisetron, palonosetron, dronabinol, aprepitant, ramosetron, metopimazine, nabilone, tropisetron, metoclopramide, prochlorperazine, trimethobenzamide, dimenhydrinate, prochlorperazine and dolasetron. The excluded active drug may be a 5HT3 antagonist selected from the group consisting of alosetron, ondansetron, granisetron, palonosetron, ramosetron and tropisetron. The excluded active drug may be a selective serotonin reuptake inhibitor selected from the group consisting of fluoxetine, sertraline, paroxetine, fluvoxamine, citalopram and alaproclate. The excluded active drug may be an anti-epileptic selected from the group consisting of carbamazepine, clonazepam, diazepam, divalproex sodium, fosphenytoin, gabapentin, lamotrigine, levetiracetam, oxcarbazepine, phenytoin, pregabalin, primidone, tiagabine, topiramate, valproate sodium, vigabatrin and zonisamide. The excluded active drug may be an anti-migraine selected from the group consisting of almotriptan, dihydroergotamine mesylate, eletriptan, frovatriptan, naratriptan, rizatriptan, sumatriptan and zolmitriptan. The excluded active drug may be an dopamine D1 and D2 antagonists selected from the group consisting of amisulpride, bromperidol, cabergoline, domperidone, fenoldopam, haloperidol, metoclopramide, metopimazine, pergolide mesylate, prochlorperazine, quetiapine, ropinirole hydrochloride, sulpiride, tiapride and zotepine. The excluded active drug may be a nootropic selected from the group consisting of almitrine dimesylate & raubasine, cevimeline hydrochloride, codergocrine mesylate, donepezil, galantamine, ginkgo biloba extract (EGb 761), memantine, nicergoline, piracetam, rivastigmine, sulbutiamine, tacrine and vinpocetine. The excluded active drug may be a statin selected from the group consisting of atorvastatin, cerivastatin, fluvastatin, lovastatin, pitavastatin, pravastatin, rosuvastatin and simvastatin.

In another embodiment of the present invention, the water soluble polymer may be selected from the group consisting of pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, natural gums, such as locust bean gum, carageenen gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

In another embodiment of the present invention, the water soluble polymer is pullulan.

In another embodiment of the present invention, the consumable film comprises about 40 to about 80 wt % pullulan; about 0.01 to about 4 wt % thymol; about 0.01 to about 4 wt % methyl salicylate; about 0.01 to about 4 wt % eucalyptol; and about 0.01 to about 15 wt % menthol.

In another embodiment of the present invention, the consumable film comprises about 0.01 to about 5 wt % of at least one stabilizing agent; about 0.001 to about 0.1 wt % of at least one of at least one coloring agent; about 0.1 to about 8 wt % of water; about 0.1 to about 15 wt % of at least one sweetening agent; about 0.1 to about 15 wt % of at least one flavoring agent; about 0.1 to about 4 wt % of at least one cooling agent; and about 0.1 to about 5 wt % of at least one surfactant.

In another embodiment of the present invention, the consumable film comprises at least one stabilizing agent selected from the group consisting of xanthan gum, locust bean gum and carrageenan, and said at least one sweetening agent is selected from the group consisting of saccharin, aspartame and acesulfame K.

In another embodiment of the present invention, the consumable film does not substantially adhere to itself.

In another embodiment of the present invention, the consumable film further comprises water in an amount from about 3 wt % to about 8 wt %.

In another embodiment of the present invention, a method for preparing an edible film comprising an active drug may be utilized wherein the method comprises: mixing at least one water soluble film former to provide a film-forming mixture; adding an active drug to the film forming mixture; casting the film forming mixture comprising the active drug on a substrate; and drying the cast film to provide the edible film comprising the active drug.

In another embodiment of the present invention, a method may be utilized wherein at least one surfactant is mixed into the film forming mixture.

In another embodiment of the present invention, a method may be utilized wherein the drying is conducted until the film has a moisture content of about 3 wt % to about 8 wt %.

In another embodiment of the present invention, a method may be utilized wherein the film-forming mixture is a powder, which is directly combined with an aqueous solution comprising an active drug to form a hydrated polymer gel.

In another embodiment of the present invention, a method may be utilized wherein the hydrated polymer gel is formed without heating.

In another embodiment of the present invention, a method may be utilized wherein the hydrated polymer gel is stirred at room temperature for about 2 to about 48 hours.

In another embodiment of the present invention, a method may be utilized comprising a non-self-adhering film comprising an active drug wherein the method comprises: mixing at least one water soluble film former to provide a film-forming mixture; adding an active drug to the film forming mixture; casting the film forming mixture comprising the active drug on a substrate; and drying the cast film to provide the edible film comprising the active drug.

In another embodiment of the present invention, a method may be utilized comprising an edible film wherein the water soluble film former is selected from the group consisting of pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, natural gums, such as locust bean gum, carageenen gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

In another embodiment of the present invention, a method may be utilized comprising an edible film wherein said water soluble polymer is pullulan.

In another embodiment of the present invention, the compositions may comprise a consumable film comprising an active drug adapted to dissolve in the mouth of a patient, wherein the film comprises an active drug in a single layer including pullulan and at least one additional active drug.

In another embodiment of the present invention, the compositions may comprise a consumable film wherein the active drug may be an anti-microbial agent selected from the group consisting of triclosan, cetyl pyridium chloride, domiphen bromide, quaternary ammonium salts, zinc compounds, sanguinarine, fluorides, alexidine, octonidine, EDTA and mixtures thereof. The active drug may be an non-steroidal anti-inflammatory agents selected from the group consisting of aspirin, acetaminophen, ibuprofen, diflunisal, fenoprofen calcium, naproxen, tolmetin sodium, indomethacin, and mixtures thereof. The active drug may be an anti-tussive selected from the group consisting of benzonatate, caramiphen edisylate, dextromethorphan hydrobromide, chlophedianol hydrochloride and mixtures thereof. The active drug may be a decongestant selected from the group consisting of pseudoephedrine hydrochloride, phenylepherine, phenylpropanolamine and mixtures thereof. The active drug may be an anti-histamine selected from the group consisting of brompheniramine maleate, chlorpheniramine maleate, carbinoxamine maleate, clemastine fumarate, dexchlorpheniramine maleate, diphenhydramine hydrochloride, diphenhydramine citrate, diphenylpyraline hydrochloride, doxylamine succinate, promethazine hydrochloride, pyrilamine maleate, tripelennamine citrate, triprolidine hydrochloride and mixtures thereof. The active drug may be an expectorant selected from the group consisting of guaifenesin, ipecac, potassium iodide, terpin hydrate and mixtures thereof. The active drug may be an anti-diarrheal wherein the anti-diarrheal is loperamide. The active drug may be an H2-antagonist selected from the group consisting of famotidine, ranitidine and mixtures thereof. The active drug may be a selective serotonin reuptake inhibitor selected from the group consisting of fluoxetine, sertraline, paroxetine, fluvoxamine, citalopram and alaproclate. The active drug may be an proton pump inhibitor selected from the group consisting of omeprazole, lansoprazole, and mixtures thereof. The active drug may be a central nervous system agent. The active drug may be an analgesic. The active drug may comprise mixtures thereof.

In another embodiment of the present invention, a method may be utilized for delivering an effective amount of an active drug to the oral cavity comprising introducing in the oral cavity a rapidly dissolving edible film comprising pullulan and an active drug.

In another embodiment of the present invention, a method may be utilized for delivering an effective amount of an active drug to the oral cavity wherein the amount of pullulan in the film is from about 40 wt % to about 80 wt %.

In another embodiment of the present invention, a method may be utilized for delivering an effective amount of an active drug to the oral cavity wherein the amount of theactive drug in the film is from about 0.0001 wt % to about 90 wt %.

In another embodiment of the present invention, a method may be utilized for delivering an effective amount of an active drug to the oral cavity comprising introducing in the oral cavity a consumable film.

In another embodiment of the present invention, the compositions may comprise an edible film comprising an active drug for use in transmucosal delivery of the active drug to a patient, wherein the film comprises a binding agent which is dissolvable in the mouth of the patient; and, a pharmacologically effective dose of an active drug dispersed in the binding agent to form a mixture that is fashioned into a film such that when the film dissolves in the mouth of the patient, the pharmacologically effective dose of the active drug is released.

### DETAILED DESCRIPTION

The present invention relates to the composition and methods of manufacture of orally-dissolvable, edible or consumable films as a vehicle for the non-invasive administration of nitroglycerin or other active drugs through the mucosal tissues of the oral cavity including, but not limited to, the mouth, pharynx, and esophagus.

In the context of the present invention, the term “active drug” includes any compound intended to furnish pharmacological activity or other direct effect in the diagnosis, cure, mitigation, treatment and/or prevention of a condition. See 21 C.F.R. 210.3(b)(7). Further, “active drugs” include those compounds of the composition that may undergo chemical change during the manufacture of the composition and be present in the final composition in a modified form intended to furnish an activity or effect. Id. In a specific embodiment of the present invention, the term “active drugs” refers to nitroglycerine and includes those compounds selected from the group consisting of anti-emetics, 5HT3 antagonists, selective serotonin reuptake inhibitors, anti-epileptics, anti-migraines, dopamine D1 and D2 antagonists, nootropics, and statins, as described in detail herein.

One embodiment of the present invention is a physiologically acceptable film that is particularly well adapted to dissolve in a mouth of a patient to deliver a nitroglycerin or other agent that can be used as an effective tool in the treatment or prevention of diseases or conditions including, but not limited to, angina pectoris, ventricular arrhythmia, supraventricular arrhythmia, and other cardio-vascular conditions and diseases, or any other disease or condition that may be treated with nitroglycerin; or, for treatment or prevention of other diseases such as those related to disorders of the gastro-intestinal or central nervous system. This film may comprise any edible or consumable polymer or film forming agent and nitroglycerin and/or another active drug.

U.S. Pat. No. 5,518,902 to Ozaki et al. (Hayashibara), the entire contents of which are incorporated by reference herein, discloses high pullulan content products, such as edible films, dentifrices and pharmaceuticals (column 3, lines 44-56 and Example B-8). The products can include a variety of ingredients in addition to pullulan, such as other polysaccharides, polyhydric alcohols, antiseptics and flavor-imparting agents (column 4, line 58 to column 5, line 11). No mention is made of delivery of nitroglycerin or other of the active drugs described herein.

U.S. Pat. No. 5,411,945 to Ozaki et al. (Hayashibara), the entire contents of which are incorporated by reference herein, discloses a pullulan binder and products produced therewith, including edible films (Example B-2). The products can include a variety of ingredients in addition to pullulan, such as other polysaccharides, antibacterial agents, flavor-imparting agents and pharmaceutically active substances (column 4, lines 5-15). No mention is made of delivery of nitroglycerin or other of the active drugs described herein.

U.S. Pat. No. 4,851,394 to Kubodera, the entire contents of which are incorporated by reference herein, discloses glucomannan/polyhydric alcohol edible films, which can comprise pullulan (column 3, line 59 to column 4, line 21). The films are contrasted with existing pullulan-based films, which are said to lack resistance to water (column 1, lines 40-44). No mention is made of delivery of nitroglycerin or other of the active drugs described herein.

U.S. Pat. No. 3,784,390 Hijiya et al., the entire contents of which are incorporated by reference herein, discloses pullulan films and their use in coating and packing materials for foods, pharmaceuticals and other oxygen sensitive materials. All of the examples in this patent teach mixing pullulan in hot water. No mention is made of delivery of nitroglycerin or other of the active drugs described herein.

U.S. Pat. No. 4,623,394 Nakamura et al., the entire contents of which are incorporated by reference herein, discloses a gradually disintegrable molded article that can be a film made with pullulan. The articles contain a particular heteromannan, which can be locust bean gum. No mention is made of delivery of nitroglycerin or other of the active drugs described herein.

U.S. Pat. No. 4,562,020 Hijiya et al., the entire contents of which are incorporated by reference herein, discloses a process for producing a self-supporting film of a glucan, which can be pullulan. No mention is made of delivery of nitroglycerin or other of the active drugs described herein.

U.S. Pat. No. 5,569,482 to Naga et al., the entire contents of which are incorporated by reference herein, discloses a method for the manufacture of an edible proteinaceous film from various sources of soybean protein. No mention is made of delivery of nitroglycerin or other of the active drugs described herein.

U.S. Pat No. 5,288,497 to Stanley et al., the entire contents of which are incorporated by reference herein, discloses methods of manufacture for the production and administration of lipophilic and nonlipophilic drugs capable of absorption through the mucosal tissues of the mouth, pharynx, and esophagus.

WO 03/011259, the entire contents of which are incorporated by reference herein, discloses maltodextrin edible films for release into the oral cavity. No mention is made of delivery of nitroglycerin or other of the active drugs described herein.

WO 03/043659, the entire contents of which are incorporated by reference herein, discloses an edible film comprised of a hydrocolloid film-forming agent that rapidly disintegrates when placed in the mouth to release an active drug. No mention is made of delivery of nitroglycerin or other of the active drugs described herein.

WO 02/43657, the entire contents of which are incorporated by reference herein, discloses pullulan-free edible film compositions and methods for making same. No mention is made of delivery of nitroglycerin or other of the active drugs described herein.

WO 02/02645, the entire contents of which are incorporated by reference herein, discloses a process for using cold-water soluble β-glucan to create a gel for use in numerous applications, including the formation of an edible film. No mention is made of delivery of nitroglycerin or other of the active drugs described herein.

WO 99/17753, the entire contents of which are incorporated by reference herein, discloses rapidly dissolving films for delivery of drugs to be adsorbed in the digestive tract. No mention is made of delivery of nitroglycerin or other of the active drugs described herein.

WO 98/26780, the entire contents of which are incorporated by reference herein, discloses a flat, foil, paper or wafer type presentation for the application and release of active substances in the buccal cavity. The specific active ingredient disclosed in WO 98/26780 is buprenorphine. No mention is made of delivery of nitroglycerin or other of the active drugs described herein.

WO 98/20862, the entire contents of which are incorporated by reference herein, discloses a film for use in the oral cavity that can contain a cosmetic or pharmaceutical active substance. No mention is made of delivery of nitroglycerin or other of the active drugs described herein.

WO 98/26763, the entire contents of which are incorporated by reference herein, discloses a flat, foil, paper or wafer like presentation for release of active substances into the buccal cavity. The particular active disclosed is apomorphine. No mention is made of delivery of nitroglycerin or other of the active drugs described herein.

U.S. Appl. Serial No. 2003/00080008, the entire contents of which are incorporated by reference herein, discloses a consumable film with high concentrations of anti-microbial agents and essential oils. No mention is made of delivery of nitroglycerin or other of the active drugs described herein.

U.S. Appl. Serial No. 2003/0035841, the entire of contents of which are incorporate by reference herein, discloses an edible film for use in the oral cavity, with at least three types film forming agents other than pullulan, including maltodextrins, hydrocolloids and fillers. No mention is made of delivery of nitroglycerin or other of the active drugs described herein.

Despite the existence of rapidly dissolving orally consumable films in the prior art, there remains room for improvement in such films, and in processes for making them, in particular, such films for the delivery of nitroglycerin and other active drugs.

Nitroglycerin, as referred to herein, is also known as 1,2,3-Propanetriol trinitrate, glyceryl trinitrate, glycerol nitric acid triester, nitroglycerol, trinitroglycerol, glonoine, trinitrin, blasting gelatin, blasting oil, and S.N.G., and is known by numerous commercial brand names, including, but not limited to, Adesitrin, Angibid, Angiolingual, Anginine, Angorin, Aquo-Trinitrosan, Cardamist, Coro-Nitro, Corditrine, Deponit, Diafusor, Gilucor “nitro”, GTN, Klavikordal, Lenitral, Lentonitrina, Millithrol, Minitran, Myoglycerin, Niong, Nitradisc, Nitran, Nitriderm, Nitro-Bid, Nitrobon, Nitrocap, Nitrocap TD, Nitrocine, Nitrocontin, Nitroderm TTS, Nitrodisc, Nitro-Dur, Nitrofortin, Nitro-Gesanit, Nitroglin, Nitroglyn, Nitroguard, Nitrol, Nitrolan, Nitrolande, Nitrolar, Nitro-lent, Nitrolin, Nitrolingual, Nitro Mack, Nitromel, Nitromin, Nitron, Nitronal, Nitronet, Nitrong, Nitro-Pflaster-ratiopharm, NitroPRN, Nitroquick, Nitrorectal, Nitroretard, Nitrosigma, Nitrospan, Nitrostat, Nitrotab, Nitro-Time, Nitrozell retard, Notrong, Nysconitrine, organic nitrate, organic nitrite, Percutol, Perlinganit, Perglottal, Reminitrol, Suscard, Sustac, Sustonit, Transderm-Nitro, Transiderm-Nitro, Tridil, Trinalgon, Trinitrosan and Vasoglyn.

Nitroglycerin is commercially available from a wide variety of sources specifically for pharmaceutical use, including, but not limited to, 3M Pharmaceuticals, Abbott Labs, Aventis Pharmaceuticals, Baxter Healthcare, Cellegy Pharmaceuticals, Inc., DuPont-Merck Pharmaceutical Co., F. Hoffman La-Roche, Ltd., Forest Laboratories, Inc., GlaxoSmithKline, Hoechst Marion Roussel, Kenwood Laboratories, Key Pharmaceuticals, Medley Pharmaceuticals, Merck & Co, Inc., Novartis Pharma AG, Parke-Davis, Pfizer, G. Pohl-Boskamp GmbH & Co., Rhone-Poulene Rorer Pharmaceutical, Inc., Schwartz Pharma AG, Solvay Pharma, Vortech Pharmaceuticals and Warner Lambert Company.

Pure nitroglycerin is a violent explosive which must be handled with great care. The stable form of nitroglycerin crystals melts in the temperate region of 55.4° F. (13° C.) and is extremely unstable as it thaws; liquid nitroglycerin will detonate if subjected to intense heat or percussion. Therefore, nitroglycerin is most useful when its explosive properties are controlled, often by dispersing the compound in an inert substance. Commercially available nitroglycerin is typically diluted to a concentration of about 10% by weight prior to manufacturing into an edible film of the present invention. For safety reasons, nitroglycerin is typically diluted to a concentration below 2% by weight prior to use in the methods of the present invention for making edible films. Additionally, in the present invention, it is recommended that certain protective apparel such as gowns, respirators, gloves and goggles, should be worn when working with nitroglycerin to avoid its toxic effects. The skin and mucus membranes readily absorb nitroglycerin and direct skin contact must therefore be avoided. Rapid absorption through the skin makes nitroglycerin a useful drug for the treatment of angina pectoris, but may be harmful to the healthy individual experiencing no oxygen deficiency in the myocardium.

Nitroglycerin may be prepared in aqueous form and is described in U.S. Pat. No. 4,879,308, the entire disclosure of which is incorporated by reference herein, and may also be prepared in non-polar liquid form as described in U.S. Pat. No. 5,869,082, the entire disclosure of which is incorporated by reference herein.

The following active drugs for use in the films of the present invention are known to function, although not necessarily solely, as agonists for serotonin receptor 5HT3. Alosetron, which functions predominantly as an anti-spasmodic and anti-cholinergic, is known in the art as an effective therapeutic in treating gastro-intestinal disorders, especially irritable bowel syndrome (IBS); Acid-related dyspepsia. Dolasetron, which functions predominantly as an anti-emetic, is known in the art as an effective therapeutic in treating gastro-intestinal disorders, especially emesis, chemotherapy-induced, surgery-induced. Granisetron, which functions predominantly as an anti-emetic, is known in the art as an effective therapeutic in treating gastro-intestinal disorders, especially emesis, chemotherapy-induced, radiation-induced, or surgery-induced. Ondansetron, which functions predominantly as an anti-emetic, is known in the art as an effective therapeutic in treating gastro-intestinal disorders, especially emesis, chemotherapy-induced, radiation-induced, and surgery-induced. Palonosetron, which functions predominantly as an anti-emetic, is known in the art as an effective therapeutic in treating gastro-intestinal disorders, especially emesis, chemotherapy-induced or surgery-induced. Ramosetron, which functions predominantly as an anti-emetic, is known in the art as an effective therapeutic in treating gastro-intestinal disorders, especially emesis, chemotherapy-induced; or due to irritable bowel syndrome (IBS). Tropisetron, which functions predominantly as an anti-emetic, is known in the art as an effective therapeutic in treating gastro-intestinal disorders, especially emesis, chemotherapy-induced.

The following active drugs for use in the films of the present invention are known to function, although not necessarily solely, as selective serotonin reuptake inhibitors. Fluoxetine, which functions predominantly as an antidepressant, is known in the art as an effective therapeutic to treat severe depression. Sertraline, which functions predominantly as an antidepressant, is known in the art as a therapeutic to treat depression, panic attacks, obsessive compulsive disorders, post-traumatic stress disorder and social anxiety disorder. Paroxetine, which functions predominantly as an antidepressant, is known in the art as a therapeutic to treat depression, panic attacks and anxiety disorders. Fluvoxamine is most often used to treat obsessive-compulsive disorder. Citalopram, which functions predominantly as an antidepressant, is known in the art as a therapeutic to treat depression, eating disorders and other mental conditions such as obsessive-compulsive disorder and panic disorder. Alaproclate is known in the art as a therapeutic to treat depression.

The following active drugs for use in the films of the present invention are known to function, although not necessarily solely, as anti-emetics. Aprepitant, a neurokinin-1 antagonist, is known in the art as an effective therapeutic in treating gastro-intestinal disorders, especially emesis, chemotherapy-induced, surgery-induced, or related to depression. Dimenhydrinate, an anti-histamine, is known in the art as an effective therapeutic in treating gastro-intestinal disorders, especially emesis. Dronabinol, a cannabinoid, is known in the art as an effective therapeutic in treating gastro-intestinal disorders, especially emesis, chemotherapy-induced, related to cachexia (wasting, AIDS related), migraines, and multiple sclerosis (MS). Metoclopramide, a dopamine D2 antagonist, is known in the art as an effective therapeutic in treating gastro-intestinal disorders, especially emesis. Metopimazine, a dopamine D2 antagonist, is known in the art as an effective therapeutic in treating gastro-intestinal disorders, especially emesis. Nabilone, a cannabinoid, is known in the art as an effective therapeutic in treating gastro-intestinal disorders, especially emesis, chemotherapy-induced. Prochlorperazine, a dopamine D2 antagonist, is known in the art as an effective therapeutic in treating gastro-intestinal disorders, especially emesis. Trimethobenzamide, an anti-emetic, is known in the art as an effective therapeutic in treating gastro-intestinal disorders, especially emesis, such as that induced by surgery.

The following active drugs for use in the films of the present invention are known in the art to function, although not solely, as anti-epileptics. Carbamazepine, an iminostilbene, is known in the art as an effective therapeutic in treating central nervous system disorders, especially epilepsy; pain, neuropathic. Clonazepam, a benzodiazepine, is known in the art as an effective therapeutic in treating central nervous system disorders, especially epilepsy; panic attacks. Diazepam, a benzodiazepine, is known in the art as an effective therapeutic in treating central nervous system disorders, especially epilepsy. Divalproex sodium, a GABA agonist, is known in the art as an effective therapeutic in treating central nervous system disorders, especially epilepsy; bipolar disorder; migraines. Fosphenytoin, an anti-convulsant, is known in the art as an effective therapeutic in treating central nervous system disorders, especially epilepsy; acute stroke. Gabapentin, a GABA agonist, is known in the art as an effective therapeutic in treating central nervous system disorders, especially epilepsy; pain, especially neuropathic; osteoarthritis. Lamotrigine, a sodium channel antagonist, is known in the art as an effective therapeutic in treating central nervous system disorders, especially epilepsy; Lennox-Gestaut syndrome; bipolar disorder; schizophrenia; pain, neuropathic; diabetic neuropathy. Levetiracetam, a pyrrolidone, is known in the art as an effective therapeutic in treating central nervous system disorders, especially epilepsy; pain, neuropathic; generalised anxiety; bipolar disorder; migraine; Parkinson's disease; social anxiety disorder. Oxcarbazepine, an iminostilbene, is known in the art as an effective therapeutic in treating central nervous system disorders, especially epilepsy; pain, neuropathic. Phenytoin, an anti-convulsant, is known in the art as an effective therapeutic in treating central nervous system disorders, especially epilepsy. Pregabalin, an alpha 2 delta ligand, is known in the art as an effective therapeutic in treating central nervous system disorders, especially pain, neuropathic; diabetic neuropathy; epilepsy; generalised anxiety; fibromyalgia; panic attacks; social anxiety disorder. Primidone, an anti-convulsant, is known in the art as an effective therapeutic in treating central nervous system disorders, especially epilepsy. Tiagabine, a GABA reuptake inhibitor, is known in the art as an effective therapeutic in treating central nervous system disorders, especially epilepsy; generalised anxiety; post-traumatic stress disorder; pain, neuropathic; insomnia. Topiramate, a sulphamate, is known in the art as an effective therapeutic in treating central nervous system disorders, especially epilepsy; Lennox-Gestaut syndrome; migraine; obesity; pain, neuropathic; hypomania; diabetic neuropathy. Valproate sodium, a GABA agonist, is known in the art as an effective therapeutic in treating central nervous system disorders, especially epilepsy; bipolar disorder. Vigabatrin, a GABA transaminase inhibitor, is known in the art as an effective therapeutic in treating central nervous system disorders, especially epilepsy. Zonisamide, a sulphonamide, is known in the art as an effective therapeutic in treating central nervous system disorders, especially epilepsy; migraine; depression; pain, neuropathic; Parkinson's disease.

The following active drugs for use in the films of the present invention are all known agonists for serotonin receptors 5HT1B and 1D, are known as effective therapeutic agents in treating disorders of the central nervous system, for example, migraine, and are especially useful as an active ingredient in anti-migraine preparations: almotriptan, dihydroergotamine mesylate, eletriptan, frovatriptan, naratriptan, rizatriptan, sumatriptan and zolmitriptan.

The following active drugs for use in the films of the present invention are all known as antagonists of the dopamine D1, D2, and/or D3 receptors. Amisulpride, a dopamine D2 and D3 antagonist which functions predominantly as an anti-psychotic, is known in the art as an effective therapeutic in treating central nervous system disorders, especially schizophrenia; depression. Bromperidol, a dopamine D2 antagonist which functions predominantly as an anti-psychotic, is known in the art as an effective therapeutic in treating central nervous system disorders, especially schizophrenia. Cabergoline, a dopamine D2 agonist which functions predominantly as an anti-Parkinson's agent, is known in the art as an effective therapeutic in treating central nervous system disorders, especially Parkinson's disease; hyperprolactinaemia. Domperidone, a dopamine D2 antagonist which functions predominantly as an anti-spasmodic and anti-cholinergic, is known in the art as an effective therapeutic in treating gastro-intestinal disorders, especially constipation; emesis; diabetic complications. Haloperidol, a dopamine D1 and D2 agonist which functions predominantly as an anti-psychotic, is known in the art as an effective therapeutic in treating central nervous system disorders, especially psychosis, acute, and/or schizophrenia. Pergolide mesylate, a dopamine D2 agonist which functions predominantly as an anti-Parkinson's agent, is known in the art as an effective therapeutic in treating central nervous system disorders, especially Parkinson's disease. Quetiapine, a dopamine D2 and 5HT2 agonist which functions predominantly as an anti-psychotic, is known in the art as an effective therapeutic in treating central nervous system disorders, especially schizophrenia; bipolar disorder; generalised anxiety; depression; Parkinson's disease; senile dementia; and Alzheimer's disease. Ropinirole hydrochloride, a dopamine D2 agonist which functions predominantly as an anti-Parkinson's agent, is known in the art as an effective therapeutic in treating central nervous system disorders, especially Parkinson's disease; restless leg syndrome; fibromyalgia. Sulpiride, a dopamine D2 agonist which functions predominantly as an antacid and anti-ulcerant, is known in the art as an effective therapeutic in treating gastro-intestinal disorders, especially schizophrenia; ulcers, gastric. Tiapride, a dopamine D2 antagonist which functions predominantly as an anti-psychotic, is known in the art as an effective therapeutic in treating central nervous system disorders, especially psychosis, acute. Zotepine, a dopamine D1, D2 and 5HT antagonist which functions predominantly as an anti-psychotic, is known in the art as an effective therapeutic in treating central nervous system disorders, especially schizophrenia. Fenoldopam, a dopamine D1 agonist which functions predominantly as an anti-hypertensive, is known in the art as an effective therapeutic in treating cardiovascular disorders, especially hypertension (HTN); congestive heart failure (CHF); renal failure, acute.

The following active drugs for use in the films of the present invention are all known in the art to function, although not necessarily solely, as nootropics. Almitrine dimesylate and raubasine, alpha 1 antagonists, are known in the art as an effective therapeutic in treating central nervous system disorders, especially senile dementia. Cevimeline hydrochloride, a cholinergic agonist, is known in the art as an effective therapeutic in treating central nervous system disorders, especially Sjogren's syndrome; radiotherapy-induced side effects. Codergocrine mesylate, an ergot alkaloid, is known in the art as an effective therapeutic in treating central nervous system disorders, especially dementia, senile. Ddonepezil, an acetylcholinesterase inhibitor, is known in the art as an effective therapeutic in treating central nervous system disorders, especially Alzheimer's disease; Parkinson's disease; dementia, cerebrovascular; migraine; attention deficit disorder/hyperactivity (ADD/ADHD). Galantamine, an acetylcholinesterase inhibitor, is known in the art as an effective therapeutic in treating central nervous system disorders, especially Alzheimer's disease; dementia, including cerebrovascular and senile. Ginkgo biloba extract (EGb 761), a memory enhancer, is known in the art as an effective therapeutic in treating central nervous system disorders, especially dementia, cerebrovascular; peripheral neuropathy; vertigo; other ear disorders; general eye disorders. Memantine, an NMDA antagonist, is known in the art as an effective therapeutic in treating central nervous system disorders, especially Alzheimer's disease; pain, neuropathic; transient ischaemic attacks (TIAs); dementia, including senile and cerebrovascular. Nicergoline, an alpha 1 antagonist, is known in the art as an effective therapeutic in treating central nervous system disorders, especially dementia, senile. Piracetam, an acetylcholine enhancer, is known in the art as an effective therapeutic in treating central nervous system disorders, especially Alzheimer's disease; dyslexia; traumatic brain injury; stroke, acute; vertigo; muscle spasticity. Rivastigmine, an acetylcholinesterase inhibitor, is known in the art as an effective therapeutic in treating central nervous system disorders, especially Alzheimer's disease; Parkinson's disease; dementia, cerebrovascular. Sulbutiamine, a psychostimulant, is known in the art as an effective therapeutic in treating central nervous system disordersand for any of various neurological indications. Tacrine, a cholinergic agonist, is known in the art as an effective therapeutic in treating central nervous system disorders, especially Alzheimer's disease. Vinpocetine, an oxygen enhancer, is known in the art as an effective therapeutic in treating central nervous system disorders, especially dementia, cerebrovascular or senile.

The following active drugs for use in the films of the present invention are all known statins/HMG CoA reductase inhibitors which function, although not necessarily solely, as anti-hyperlipidaemics. Atorvastatin is known in the art as an effective therapeutic in treating cardiovascular disorders, especially hyperlipidaemia and atherosclerosis. Cerivastatin is known in the art as an effective therapeutic in treating cardiovascular disorders, especially hyperlipidaemia; diabetes, type II (maturity onset); stroke prophylaxis; atherosclerosis; coronary artery disease (CAD); menopause; myocardial infarction, acute (AMI); renal insufficiency. Fluvastatin, is known in the art as an effective therapeutic in treating cardiovascular disorders, especially hyperlipidaemia; atherosclerosis; angioplasty complications, prevention. Lovastatin, is known in the art as an effective therapeutic in treating cardiovascular disorders, especially hyperlipidaemia; atherosclerosis; myocardial infarction prophylaxis; angina, unstable; coronary artery bypass graft (CABG); and Alzheimer's disease. Pitavastatin, is known in the art as an effective therapeutic in treating cardiovascular disorders, especially hyperlipidaemia. Pravastatin, is known in the art as an effective therapeutic in treating cardiovascular disorders, especially hyperlipidaemia; atherosclerosis; stroke prophylaxis. Rosuvastatin, is known in the art as an effective therapeutic in treating cardiovascular disorders, especially hyperlipidaemia; atherosclerosis. Simvastatin, is known in the art as an effective therapeutic in treating cardiovascular disorders, especially hyperlipidaemia; transient ischaemic attacks (TIAs); myocardial infarction prophylaxis; myocardial infarction, acute (AMI).

In another embodiment of the present invention, the compositions of the present invention may exclude an active drug comprising one or more anti-emetics. Such anti-emetics include and may be selected from one or more of the group consisting of: ondansetron, granisetron, palonosetron, dronabinol, aprepitant, ramosetron, metopimazine, nabilone, tropisetron, metoclopramide, prochlorperazine, trimethobenzamide, dimenhydrinate, prochlorperazine and dolasetron.

In another embodiment of the present invention, compositions of the present invention may exclude an active drug comprising one or more 5HT3 antagonists. Such 5HT3 antagonists include and may be selected from one or more of the group consisting of: alosetron, ondansetron, granisetron, palonosetron, ramosetron and tropisetron.

In another embodiment of the present invention, compositions of the present invention may exclude an active drug comprising one or more selective serotonin reuptake inhibitors. Such selective serotonin reuptake inhibitors may be selected from one or more of the group consisting of: fluoxetine, sertraline, paroxetine, fluvoxamine, citalopram and alaproclate.

In another embodiment of the present invention, the compositions of the present invention may exclude an active drug comprising one or more anti-epileptics. Such anti-epileptics include and may be selected from one or more of the group consisting of: carbamazepine, clonazepam, diazepam, divalproex sodium, fosphenytoin, gabapentin, lamotrigine, levetiracetam, oxcarbazepine, phenytoin, pregabalin, primidone, tiagabine, topiramate, valproate sodium, vigabatrin and zonisamide.

In another embodiment of the present invention, the compositions of the present invention may exclude an active drug comprising one or more anti-migraines. Such anti-migraines include and may be selected from one or more of the group consisting of: almotriptan, dihydroergotamine mesylate, eletriptan, frovatriptan, naratriptan, rizatriptan, sumatriptan and zolmitriptan.

In another embodiment of the present invention, the compositions of the present invention may exclude an active drug comprising one or more dopamine D1 and D2 antagonists. Such dopamine D1 and D2 antagonists include and may be selected from one or more of the group consisting of: amisulpride, bromperidol, cabergoline, domperidone, fenoldopam, haloperidol, metoclopramide, metopimazine, pergolide mesylate, prochlorperazine, quetiapine, ropinirole hydrochloride, sulpiride, tiapride and zotepine.

In another embodiment of the present invention, the compositions of the present invention may exclude an active drug comprising one or more nootropics. Such nootropics include and may be selected from one or more of the group consisting of: almitrine dimesylate & raubasine, cevimeline hydrochloride, codergocrine mesylate, donepezil, galantamine, ginkgo biloba extract (EGb 761), memantine, nicergoline, piracetam, rivastigmine, sulbutiamine, tacrine and vinpocetine.

In another embodiment of the present invention, the compositions of the present invention may exclude an active drug comprising one or more statins. Such statins include and may be selected from one or more of the group consisting of: atorvastatin, cerivastatin, fluvastatin, lovastatin, pitavastatin, pravastatin, rosuvastatin and simvastatin.

In another embodiment of the invention, the film may exclude one or more of the anti-emetics, 5HT3 antagonists, anti-epileptics, anti-migraines, dopamine D1 and D2 antagonists, nootropics and statins listed above and including others known in the art.

In another embodiment of the present invention, the compositions within the film may exclude chemical combinations that create adverse drug interactions. The phrase “chemical combinations” is employed herein to refer to active drugs, compounds, materials such as flavorants, excipients, compositions, and/or dosage forms, which are, within the scope of sound medical judgment, suitable as combinations for use in contact with the tissues of a human being or consumption from a human being without excessive toxicity, irritation, allergic response, or other problem or complication commensurate with a reasonable benefit/risk ratio. For example, the combination of paroxetine or other selective serotonin reuptake inhibitors with St. John's Wort is reported to cause adverse side effects such as confusion, nausea, weakness, and/or fatigue. In one specific embodiment of the present invention, compositions wherein a consumable or edible film comprises paroxetine may specifically exclude St. John's Wort.

### Composition of Films

An embodiment of the invention is a fast dissolving film that comprises a physiologically acceptable amount of nitroglycerin. The expression “physiologically acceptable” amounts of nitroglycerin, as used herein, is intended to encompass an amount or dose, which upon administration to a patient, is adequately tolerated and effective for treatment without causing undue negative side effects, and are physiologically acceptable and compatible with oral films. The amount of nitroglycerin that can be used in the rapidly dissolving films, according to the present invention, is dependent upon the dose needed to provide an effective amount of nitroglycerin.

The dosage needed to provide an effective amount of nitroglycerin may be readily determined by one of ordinary skill in the art using well known techniques, and is typically an amount that will cause an amelioration of symptoms or disease. Specific doses may be adjusted depending on conditions of the disease, the age, body weight, general health, sex, diet of the subject, dose intervals, excretion rate and combinations with other drugs. As used herein, a therapeutically effective amount of nitroglycerin is an amount in the range of about 0.001 mg to about 1000 mg, or in the range of about 0.01 mg to about 100 mg, or in the range of about 0.05 mg to about 50 mg, or in the range of about 0.1 mg to about 40 mg.

### Preparation of Films

The active drug comprising film of the present invention in one embodiment comprises at least one film-forming agent and may further comprise water, additional film-forming agents, triglycerides, preservatives, polyethylene oxide compounds, propylene glycol, potentiating agents, saliva stimulating agents, plasticizing agents, cooling agents, surfactants, nitroglycerin stabilizing agents, film stabilizing agents, emulsifying agents, thickening agents, binding agents, buffers, releasing agents, permeation enhancers, sweeteners, additional natural and artificial flavoring agents, coloring agents, coating agents, additional pharmaceutically active agents, antibacterial agents, antiviral agents, and the like.

The film-forming agent used in the films according to the present invention can be any suitable film-forming agent including, but not limited to, pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, natural gums, such as locust bean gum, carrageenan gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

In one embodiment of the present invention, at least one film former is pullulan, in amounts ranging from about 0.01 to about 99 wt %, about 30 to about 80 wt %, or from about 45 to about 70 wt % of the film, or from about 60 to about 65 wt % of the film.

In yet another embodiment of the present invention, at least one film former is a hydrocolloid material known in the art for its film-forming properties. The hydrocolloid material may be present in a wide range of concentrations, including, but not limited to, amounts ranging from about 50 to about 90 wt %, or at about 50 to about 80 wt %.

In another embodiment of the present invention, at least one film former is a maltodextrin. The maltodextrin may be present in a wide range of concentrations, including but not limited to, amounts ranging from between about 5 to about 60 wt %, preferably between about 20 to about 40 wt %, and may be present with a hydrocolloid material, in a range of between about 10 to about 50 wt %, or about 30 to about 40 wt % of the film.

In yet another embodiment of the present invention, at least one film former is a purified β-glucan solution. The β-glucan solution may be used in a wide range of concentrations, including but not limited to a range of about 10 wt % of the film.

The films comprising active drugs also may include a triglyceride. Examples of triglycerides include, but are not limited to, vegetable oils such as corn oil, sunflower oil, peanut oil, olive oil, canola oil, soybean oil and mixtures thereof. In one embodiment, the triglyceride is olive oil. The triglyceride is added to the film in amounts from about 0.1 wt % to about 12 wt %, or in a range from about 0.5 wt % to about 9 wt %, of the film.

The films comprising active drugs also may include a preservative. The preservative may be added in amounts from about 0.001 wt % to about 5 wt %, or from about 0.01 wt % to about 1 wt % of the film. In one embodiment, preservatives include sodium benzoate and potassium sorbate.

The films comprising active drugs may also include a polyethylene oxide compound. The molecular weight of the polyethylene oxide compound may be within a very broad range, including but not limited to ranges from about 50,000 to about 6,000,000. In one embodiment, the polyethylene oxide compound is N-10 available from Union Carbide Corporation. The polyethylene oxide compound may be added in amounts from about 0.1 wt % to about 5 wt %, or from about 0.2 wt % to about 4.0 wt % of the film.

The films comprising active drugs may also include propylene glycol. The propylene glycol may be added in wide range of amounts, including but not limited to from about 1 wt % to about 20 wt %, or from about 5 wt % to about 15 wt % of the film.

The films comprising active drugs may also include a nitroglycerin potentiating agent. Such nitroglycerin potentiating agents include, but are not limited to, menthol, as disclosed in U.S. Pat. No. 6,559,180, the entire content of which is incorporate by reference herein.

The films comprising active drugs also may include saliva stimulating agents. Useful saliva stimulating agents include, but are not limited to, those disclosed in U.S. Pat. No. 4,820,506, which is incorporated by reference herein. Saliva stimulating agents include food acids such as citric, lactic, malic, succinic, ascorbic, adipic, fumaric and tartaric acids. Suitable food acids include, but are not limited to, citric, malic and ascorbic acids. The amount of saliva stimulating agents in the film may be used in a wide range of amounts, including but not limited to from about 0.01 to about 12 wt %, or about 1 wt % to about 10 wt %, or about 2.5 wt % to about 6 wt %.

Plasticizing agents including, but not limited to, triacetin may be added to the films comprising active drugs in a wide range of amounts, including but not limited to amounts ranging from about 0 to about 20 wt %, or about 0 to about 2 wt %. Other suitable plasticizing agents include, but are not limited to, polyols, such as sorbitol, glycerin, polyethylene glycol, propylene glycol, hydrogenated starch hydrolysates, corn syrups, as well as monoacetin, diacetin, maltitol and mannitol.

Cooling agents including, but not limited to, monomenthyl succinate may be added to the films comprising active drugs in a wide range of amounts, including but not limited to amounts ranging from about 0.001 to about 2.0 wt %, or about 0.2 to about 0.4 wt %. A monomenthyl succinate containing cooling agent is available from Mane, Inc. Other suitable cooling agents include, but are not limited to, WS3, WS23, Ultracool II and the like.

Surfactants including, but not limited to, mono and diglycerides of fatty acids and polyoxyethylene sorbitol esters, such as, Atmos 300 and Polysorbate 80 may be added to the films comprising active drugs. The surfactant may be added in a wide range of amounts, including but not limited to amounts ranging from about 0.5 to about 15 wt %, or about 1 to about 5 wt % of the film. Other suitable surfactants include, but are not limited to, pluronic acid, sodium lauryl sulfate, and the like.

The films comprising active drugs may also include a stabilizer in the film. The presence of a stabilizer in the film decreases the loss of active drugs in the film and may prolong shelf-life as well. For example, suitable stabilizers for the active drug nitroglycerin are known in the art, and include, but are not limited to, glyceryl monostearate, which is described in U.S. Pat. No. 6,500,456, the entire content of which is incorporated by reference herein.

Film stabilizing agents including, but not limited to, xanthan gum, locust bean gum and carrageenan, in a wide range of amounts including but not limited to amounts ranging from about 0 to about 10 wt %, or about 0.1 to about 2 wt %, may be added to the films comprising active drugs. Other suitable stabilizing agents include, but are not limited to, guar gum and the like.

Emulsifying agents including, but not limited to, lecithin, bentonite, veegum, stearates, triethanolamine stearate, ester derivatives of stearates, palmitates, ester derivatives of palmitates, oleates, ester derivatives of oleates, glycerides, ester derivatives of glycerides, sucrose polyesters, polyglycerolesters, animal waxes, vegetable waxes, synthetic waxes, petroleum, quaternary ammonium compounds, acacia, gelatin, and the like may be added to the films comprising active drugs in a wide range of amounts, including but not limited to amounts ranging from about 0 to about 5 wt %, or about 0.01 to about 0.7 wt % of the film.

Thickening agents including, but not limited to, cellulose ethers, such as methylcellulose, carboxyl methylcellulose, and the like may be added to the films comprising active agents in a wide range of amounts, including but not limited to amounts ranging from about 0 to about 20 wt %, or about 0.01 to about 5 wt %.

Binding agents including, but not limited to, starch may be added to the films comprising active drugs in a wide range of amounts, including but not limited to amounts ranging from about 0 to about 10 wt %, or about 0.01 to about 2 wt % of the film.

Suitable sweeteners may be included in the films comprising active drugs include those well known in the art, including both natural and artificial sweeteners. Suitable sweeteners include, but are not limited to: water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin; water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2, 2-dioxide, the potassium salt of 3,4-dihydro-6-methyl-1,2,3-oxathiazine-4-one-2,2-dioxide (acesulfame-K), the free acid form of saccharin, and the like; dipeptide based sweeteners, such as L-aspartic acid derived sweeteners, such as L-aspartyl-L-phenylalanine methyl ester (aspartame) and materials described in U.S. Pat. No. 3,492,131, which is incorporated by reference herein, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like; water-soluble sweeteners derived from naturally occurring water-soluble sweeteners, such as a chlorinated derivative of ordinary sugar (sucrose), known, for example, under the product description of sucralose; and protein based sweeteners such as thaumatoccous danielli (Thaumatin I and II).

In general, an effective amount of auxiliary sweetener is utilized to provide the level of sweetness desired for a particular composition, and this amount will vary with the sweetener selected. This amount will normally be 0.01% to about 10% by weight of the composition when using an easily extractable sweetener. The water-soluble sweeteners described in paragraph [00116] above, are usually used in amounts of about 0.01 to about 10 wt %, and preferably in amounts of about 2 to about 5 wt %. Some of the sweeteners in paragraph [00116] (e.g., glycyrrhizin) can be used in amounts set forth for paragraphs [00117]-[00120] below due to the sweeteners' known sweetening ability. In contrast, the sweeteners described in paragraphs [00117]-[00120]are generally used in amounts of about 0.01 to about 10 wt %, or about 2 to about 8 wt %, or about 3 to about 6 wt %. These amounts may be used to achieve a desired level of sweetness independent from the flavor level achieved from any optional flavor oils used.

The active drugs used in the film can be coated to mask the taste of active drugs or to prevent the active drugs from numbing the tongue or other surfaces in the oral cavity. The coatings that can be used are known to those skilled in the art. These include, but are not limited to, polymers such, as Eudragit® E, cellulosics, such as ethylcellulose, and the like. An additional way to mask the taste of active drugs may be by using an ion exchange resin such as Amberlite RP-69, available from Rohm and Haas, and Dow XYS-40010.00, available from the Dow Chemical Co.

Additional natural and artificial flavorings may be chosen from synthetic flavor oils and flavoring aromatics, and/or oils, oleo resins and extracts derived from plants, leaves, flowers, fruits and so forth, and combinations thereof. Representative flavor oils include, but are not limited to, spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to, acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde is C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof, and the like.

The amount of flavoring employed in the film comprising active drugs may be normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.1 to about 30 wt % are useable with amounts of about 2 to about 25 wt % or amounts from about 8 to about 10 wt %.

The films comprising active drugs of this invention may also contain coloring agents or colorants. The coloring agents may be used in amounts effective to produce the desired color. The coloring agents useful in the present invention, include pigments such as titanium dioxide, which may be incorporated in amounts of up to about 5 wt %, and preferably less than about 1 wt %. Colorants may also include natural food colors and dyes suitable for food, drug and cosmetic applications. These colorants are known as FD&C dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 3 comprises a triphenylmethane dye and is the monosodium salt of 4-[4-N-ethyl-p-sulfobenzylamino) diphenyl-methylene]-[1-N-ethyl-N-p-sulfonium benzyl)-2,5-cyclo-hexadienimine]. A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.

In order to prepare a desirable active drugs containing dissolvable matrix for formation into a dosage-form, it may be necessary to combine several general types of components. These components include, but are not limited to, the types of components used to prepare typical confections, the active drugs, and other desired chemically active ingredients such as buffering agents, permeation enhancers and the like.

The types of components involved may generally fall into the following categories, including but not limited to: flavorings, sweeteners, flavor enhancers, releasing agents, buffers, one or more therapeutic agents, dissolvable matrix material, and permeation enhancers. The components may be a releasable or slowly releasable liquid.

As mentioned above, these components may each be provided in a form which facilitates mixing, such as a dry powder. This provides for convenient combination of the ingredients, even if they happen to be insoluble or otherwise chemically incompatible. All or some of the incipients or inactive ingredients may be on the GRAS list (“generally regarded as safe”).

In certain medications, it may also be desirable to add a lubricating agent in order to release the dosage-form from the mold. Such agents may also provide a certain amount of waterproofing. As mentioned above, the rate of dissolution of the dosage-form within the patient's mouth may be controlled chemically, as well as physically, through the extent of compression of the composition. These lubricating or releasing agents may include, but are not limited to, substances such as compritol 888 (glyceryl behenate), calcium stearate, and sodium stearate. These agents may enhance dissolution or they may inhibit dissolution as necessary.

Lubricating agents may also be useful in those embodiments wherein a powder mixture is funneled into a chute during manufacture. Lubricating agents and surfactants may improve product flow and may avoid static electricity charge buildup within the formulation which may cause the ingredients to separate due to electrostatic forces.

It may also be desirable to include buffering agents within the composition. Buffering agents may provide the ability to place the film comprising active drugs in the mouth in a favorable pH environment for passage across the mucosal tissues of the mouth, pharynx, and esophagus. Buffering agents incorporated within the composition may be used to affect a pH change in the salival environment of the mouth in order to favor the existence of a unionized form of the active drug or other active ingredient which more readily moves through the mucosal tissues.

In addition, appropriate pH adjustment may aid in producing a more palatable product with nitroglycerin or other active drugs which are either severely acidic (and thus sour) or severely basic (and thus bitter). As a result, a buffer system such as citric acid/sodium citrate may be desirable for addition into the dissolvable matrix. A phosphate buffer system may also be used.

A suitable permeation enhancer capable of improving the drug permeability across the mucosal membrane may also be included in the dissolvable composition. Permeation enhancers may be particularly important when nonlipophilic drugs are used, but may be valuable for lipophilic drugs as well. Examples of typical permeation enhancers which may be used within the scope of the present invention, include, but are not limited to bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate, as well as sodium dodecyl sulfate (“SDS”), dimethyl sulfoxide (“DMSO”), sodium lauryl sulfate, salts and other derivatives of saturated and unsaturated fatty acids, surfactants, bile salt analogs, derivatives of bile salts. Additionally, synthetic permeation enhancers, as described in U.S. Pat. No. 4,746,508, the entire contents of which are incorporated by reference herein, may also be used.

It will be appreciated by those of ordinary skill in the art that filling and bulking agents of the type known in the art may also be used if desired in the films of the present invention, including but not limited to lactose or gelatin.

Added to the dissolvable matrix described above will be the appropriate amount of an active drug. As will be discussed in more detail below, an active drug is easily incorporated into the matrix compositions to produce the edible or consumable films comprising active drugs of the present invention.

Each of the desired components may be mixed to produce the compositions of the present invention. It may be useful, but not required, to use the method of geometric dilution in mixing the various components. Using this method, the two smallest ingredients by weight (as a proportion of the final product) are first mixed together thoroughly.

When complete mixing has been obtained between those two components, the next smallest ingredient or ingredients by weight equal to the weight of the previous ingredients is added and mixed thoroughly with the existing mixture. This procedure is repeated until all of the components are added to the mix and mixed thoroughly with all other components.

Geometric dilution provides for complete and thorough mixing of all of the components. Using the method described above, there may be less chance for incomplete mixing and uneven distribution of components throughout the mix. Other existing methods may result in incomplete mixing because of the insolubility of the products.

Once complete mixing is accomplished, the mixture may be formed into a solid dissolvable matrix composition. In one embodiment, the mixture may be compressed under relatively high forces to provide a coherent dosage. Compressive forces in the range of from approximately 2,000 Newtons to approximately 5,000 Newtons are suitable, however, any force which is sufficient to compress the ingredients into a coherent, integrated mass could be used.

In other embodiments within the scope of the present invention, the desired constituents may be formed into the dosage-form by dehydration, freeze drying (lyophilization), pouring into a mold, spraying onto a suitable holder, vapor deposition, or other known techniques in the art.

When producing the edible films comprising active drugs, there may be no need to heat the mixture to a molten mass as has been the practice in the past in forming drug-containing confections. As a result, heat degradation of active drugs may be avoided while good mixing and a uniform product may be provided.

In addition to active drugs, it is readily apparent to those of ordinary skill in the art that a variety of other active drugs can be added to the edible films of the present invention. These active drugs are not particularly limited; however, they should be physiologically acceptable and compatible with the film. Suitable active drugs include, but are not limited to: anti-microbial agents, such as triclosan, cetyl pyridium chloride, domiphen bromide, quaternary ammonium salts, zinc compounds, sanguinarine, fluorides, alexidine, octonidine, EDTA, and the like; non-steroidal anti-inflammatory drugs, such as aspirin, acetaminophen, ibuprofen, ketoprofen, diflunisal, fenoprofen calcium, naproxen, tolmetin sodium, indomethacin, and the like; anti-tussives, such as benzonatate, caramiphen edisylate, menthol, dextromethorphan hydrobromide, chlophedianol hydrochloride, and the like; decongestants, such as pseudoephedrine hydrochloride, phenylepherine, phenylpropanolamine, pseudoephedrine sulfate, and the like; anti-histamines, such as brompheniramine maleate, chlorpheniramine maleate, carbinoxamine maleate, clemastine fumarate, dexchlorpheniramine maleate, diphenhydramine hydrochloride, diphenylpyraline hydrochloride, azatadine meleate, diphenhydramine citrate, doxylamine succinate, promethazine hydrochloride, pyrilamine maleate, tripelennamine citrate, triprolidine hydrochloride, acrivastine, loratadine, brompheniramine, dexbrompheniramine, and the like;expectorants, such as guaifenesin, ipecac, potassium iodide, terpin hydrate, and the like; anti-diarrheals, such a loperamide, and the like; H2-antagonists, such as famotidine, ranitidine, and the like; selective serotonin reuptake inhibitors such as fluoxetine, sertraline, paroxetine, fluvoxamine, citalopram and alaproclate; proton pump inhibitors, such as omeprazole, lansoprazole, and the like; general nonselective CNS depressants, such as aliphatic alcohols, barbiturates and the like; general nonselective CNS stimulants such as caffeine, nicotine, strychnine, picrotoxin, pentylenetetrazol and the like; drugs that selectively modify CNS function such as phenyhydantoin, phenobarbital, primidone, carbamazepine, ethosukimide, methsuximide, phensuximide, trimethadione, diazepam, benzodiazepines, phenacemide, pheneturide, acetazolamide, sulthiame, bromide, and the like; anti-parkinsonism drugs such as levodopa, amantadine and the like; narcotic-analgesics such as morphine, heroin, hydromorphone, metopon, oxymorphone, levorphanol, codeine, hydrocodone, xycodone, nalorphine, naloxone, naltrexone and the like; analgesic-antipyretics such as salycilates, phenylbutazone, indomethacin, phenacetin and the like; psychopharmacological drugs such as chlorpromazine, methotrimeprazine, haloperidol, clozapine, reserpine, imipramine, tranylcypromine, phenelzine, lithium and the like; anti-hypertension and cardiovascular treatment agents such as ACE inhibitors, calcium channel blockers, peripheral vasodilators, beta adrenergic blockers, alpha/beta adrenergic blockers, diuretics, digitalis, and isosorbide nitrates, including isosorbide dinitrates and isosorbide mononitrates.

The active drugs in the edible or consumable films of the present invention are prepared to provide a particular dosage per portion of the film. The thickness width and length of the film may be used to calculate the dose contained in the film if the active drugs are uniformly distributed throughout at a known or predetermined concentration. Alternatively, the amount of active drugs added to the film ingredients may be adjusted to provide a desired dose of active dugs when the thickness width and length of the film are uniform.

### EXAMPLES

The invention will be illustrated in more detail with reference to the following Examples, but it should be understood that the present invention is not deemed to be limited thereto.

### Example 1

The following method is used to prepare films comprising the selective serotonin reuptake inhibitor, paroxetine; the film-forming ingredients (e.g., xanthan gum, locust bean gum, carrageenan and pullulan) other than Polysorbate 80 and Atmos 300 are mixed and hydrated in hot purified water to form a gel and stored in a refrigerator overnight at a temperature of approximately 4° C. to form preparation A; the coloring agent(s), copper gluconate and sweetener are added to and dissolved in purified water to form preparation B; preparation B is added to preparation A and mixed well to form preparation C; the flavoring agent(s) is mixed to form preparation D; the polysorbate 80 and Atmos 300 are added to preparation D and mixed well to form preparation E; and preparation E is added to preparation C and mixed well to form preparation F.

Paroxetine is added to any of the above-described preparations in the desired amount to yield the desired dosage in the finished film. Preparation F is poured on a mold and cast to form a film of a desired thickness at room temperature. The film is dried under warm air and cut to a desired dimension, packaged and stored.

### Example 2

Edible films comprising the selective serotonin reuptake inhibitor, paroxetine, are prepared using a method which comprises the following steps: dissolve copper gluconate, acesulfame K, aspartame, glycerin, sorbitol and dye in purified water to form an aqueous mixture; mix pullulan, xanthan gum, locust bean gum and carrageenan together in powder form to form a powder mixture; add the powder mixture from step B to the aqueous mixture from step A to form a hydrated polymer gel; stir the hydrated polymer from step C at slow speed (about 50-100 RPM) overnight at room temperature; cast the uniform mixture from step D on a suitable backing; and dry the cast mixture to form a film.

Paroxetine may be added to the mixture at any of Steps A through D at a desired amount to provide a desired dose of paroxetine in the finished film. The finished film is cut to the desired dimensions and stored.

It can be seen, therefore, that the present invention provides a great deal of flexibility in the construction of an appropriate drug-containing confection. The quantity of drug contained in any confection can be varied within wide ranges. In addition, various methods of attachment of the confection to the handle are available in order to provide a wide range of flexibility.

### Example 3

Edible films comprising the selective serotonin reuptake inhibitor, paroxetine, may be prepared as follows: add sodium benzoate and sweeteners to water; mix locust bean gum, xanthan gum and carrageenan together; add the gum mixture to the mixture of step 1 and mix until dissolved; mix paroxetine with either water or propylene glycol in an amount to provide the desired dose of paroxetine in the finished film; add the remaining desired ingredients to the mixture of step 4 or mix the remaining desired ingredients in a separate mixture; add the mixtures of step 4 and step 5 to the mixture of step 3; and cast and dry to make a film and cut to a size to achieve the desired paroxetine dose.

### Example 4

Edible films comprising the selective serotonin reuptake inhibitor, paroxetine, may be prepared as follows: add sodium benzoate to water heated to 50 C. Mix to dissolve; separately, add Peg 1450, titanium dioxide and paroxetine to the mixture of step 1, mixing with each addition. The amount of paroxetine added is the amount that yields the desired paroxetine dose in the finished film; mix the locust bean gum, xanthan gum and carrageenan together; add the gums to the mixture of step 2 and mix until dissolve; add the remaining ingredients together with heat if needed; and add the mixture of steps 4 and 5 together. Cast and dry to make a film and cut to a size to achieve the desired dose.

The paroxetine in the edible films of the present invention is prepared to provide a particular dosage per portion of the film. The thickness width and length of the film can be used to calculate the dose contained in the film if the paroxetine is uniformly distributed throughout at a known or predetermined concentration. Alternatively, the amount of paroxetine added to the film ingredients may be adjusted to provide a desired dose of paroxetine when the thickness width and length of the film are uniform.

### Example 5

Edible films comprising the selective serotonin reuptake inhibitor, paroxetine, may be prepared as follows: add hydrocolloid starch solution to de-ionized water with high shear mixing until clear water is formed; heat de-ionized water to 40° C. and add protein solution (e.g. fish gelatin) with slow agitation until protein is dissolved; reducing heat to 30° C.; add mixture of step 1 and step 2 with Sorbo Sorbitol solution and Polysorbate 80 and mix until dissolved; mix paroxetine with either water or propylene glycol in an amount to provide the desired dose of paroxetine in the finished film; add the remaining desired ingredients to the mixture of step 4 or mix the remaining desired ingredients in a separate mixture; and add the mixtures of step 4 and step 5 to the mixture of step 3. Cast onto a polyethylene coated differential release paper using a knife-over-roll coating head, and dry in drying tunnel to make a film and cut to a size to achieve the desired paroxetine dose.

### Example 6

Edible films comprising the selective serotonin reuptake inhibitor, paroxetine, may be prepared as follows: mix maltodextrin, sodium alginate and 10 microcrystalline cellulose to water heated to boiling while stirring; cool mixture to a temperature between 35° C. to about 40° C., adding flavor/emulsifier blends, sweeteners, softeners and color to mixture; mix paroxetine with either water or propylene glycol in an amount to provide the desired dose of paroxetine in the finished film; add the remaining desired ingredients to the mixture of step 3 or mix the remaining desired ingredients in a separate mixture; add the mixtures of step 3 and step 4 to the mixture of step 2; and spread onto a glass plate by utilizing a draw down blade, and dry solution in an oven for about 15 minutes at 40° C. to make a film and cut to a size to achieve the desired paroxetine dose.

### Example 7

Edible films comprising the selective serotonin reuptake inhibitor, paroxetine, may be be prepared as follows: mix a purified β-glucan in heated water to form a β-glucan solution; mix paroxetine with either water or propylene glycol in an amount to provide the desired dose of paroxetine in the finished film; add the remaining desired ingredients to the mixture of step 2 or mix the remaining desired ingredients in a separate mixture; pour liquid mixture onto a heated bomb at 150° C. for 15 minutes to evaporate water from solution; and peel film off hot surface and dry further in an oven at 70° C., and cut to a size to achieve the desired paroxetine dose.

What is claimed is:

1. A consumable film adapted to dissolve in a mouth of a patient, wherein said film comprises one or more active drugs and a water soluble polymer.

2. The consumable film of claim 1, wherein said one or more active drugs may be selected from the group consisting of anti-emetics, 5HT3 antagonists, selective serotonin reuptake inhibitors, anti-epileptics, anti-migraines, dopamine D1 and D2 antagonists, nootropics, and statins.

3. The active drugs of claim 2, wherein said one or more anti-emetics are selected from the group consisting of ondansetron, granisetron, palonosetron, dronabinol, aprepitant, ramosetron, metopimazine, nabilone, tropisetron, metoclopramide, prochlorperazine, trimethobenzamide, dimenhydrinate, prochlorperazine and dolasetron.

4. The active drugs of claim 2, wherein said one or more 5HT3 antagonsists are selected from the group consisting of alosetron, ondansetron, granisetron, palonosetron, ramosetron and tropisetron.

5. The active drugs of claim 2, wherein said one or more selective serotonin reuptake inhibitors are selected from the group consisting of fluoxetine, sertraline, paroxetine, fluvoxamine, citalopram and alaproclate.

6. The active drugs of claim 2, wherein said one or more anti-epileptics are selected from the group consisting of carbamazepine, clonazepam, diazepam, divalproex sodium, fosphenytoin, gabapentin, lamotrigine, levetiracetam, oxcarbazepine, phenytoin, pregabalin, primidone, tiagabine, topiramate, valproate sodium, vigabatrin and zonisamide.

7. The active drugs of claim 2, wherein said one or more anti-migraines are selected from the group consisting of almotriptan, dihydroergotamine mesylate, eletriptan, frovatriptan, naratriptan, rizatriptan, sumatriptan and zolmitriptan.

8. The active drugs of claim 2, wherein said one or more dopamine D1 and D1 antagonists are selected from the group consisting of amisulpride, bromperidol, cabergoline, domperidone, fenoldopam, haloperidol, metoclopramide, metopimazine, pergolide mesylate, prochlorperazine, quetiapine, ropinirole hydrochloride, sulpiride, tiapride and zotepine.

9. The active drugs of claim 2, wherein said one or more nootropics are selected from the group consisting of almitrine dimesylate & raubasine, cevimeline hydrochloride, codergocrine mesylate, donepezil, galantamine, ginkgo biloba extract (EGb 761), memantine, nicergoline, piracetam, rivastigmine, sulbutiamine, tacrine and vinpocetine.

10. The active drugs of claim 2, wherein said one or more statins are selected from the group consisting of atorvastatin, cerivastatin, fluvastatin, lovastatin, pitavastatin, pravastatin, rosuvastatin and simvastatin.

11. The consumable film of claim 1, wherein said one or more active drugs may be excluded, which may be selected from the group consisting of anti-emetics, 5HT3 antagonists, selective serotonin reuptake inhibitors, anti-epileptics, anti-migraines, dopamine D1 and D2 antagonists, nootropics, and statins.

12. The active drugs of claim 11, wherein said one or more anti-emetics are selected from the group consisting of ondansetron, granisetron, palonosetron, dronabinol, aprepitant, ramosetron, metopimazine, nabilone, tropisetron, metoclopramide, prochlorperazine, trimethobenzamide, dimenhydrinate, prochlorperazine and dolasetron.

13. The active drugs of claim 11, wherein said one or more 5HT3 antagonsists are selected from the group consisting of alosetron, ondansetron, granisetron, palonosetron, ramosetron and tropisetron.

14. The active drugs of claim 11, wherein said one or more selective serotonin reuptake inhibitors are selected from the group consisting of fluoxetine, sertraline, paroxetine, fluvoxamine, citalopram and alaproclate.

15. The active drugs of claim 11, wherein said one or more anti-epileptics are selected from the group consisting of carbamazepine, clonazepam, diazepam, divalproex sodium, fosphenytoin, gabapentin, lamotrigine, levetiracetam, oxcarbazepine, phenytoin, pregabalin, primidone, tiagabine, topiramate, valproate sodium, vigabatrin and zonisamide.

16. The active drugs of claim 11, wherein said one or more anti-migraines are selected from the group consisting of almotriptan, dihydroergotamine mesylate, eletriptan, frovatriptan, naratriptan, rizatriptan, sumatriptan and zolmitriptan.

17. The active drugs of claim 11, wherein said one or more dopamine D1 and D1 antagonists are selected from the group consisting of amisulpride, bromperidol, cabergoline, domperidone, fenoldopam, haloperidol, metoclopramide, metopimazine, pergolide mesylate, prochlorperazine, quetiapine, ropinirole hydrochloride, sulpiride, tiapride and zotepine.

18. The active drugs of claim 11, wherein said one or more nootropics are selected from the group consisting of almitrine dimesylate & raubasine, cevimeline hydrochloride, codergocrine mesylate, donepezil, galantamine, ginkgo biloba extract (EGb 761), memantine, nicergoline, piracetam, rivastigmine, sulbutiamine, tacrine and vinpocetine.

19. The active drugs of claim 11, wherein said one or more statins are selected from the group consisting of atorvastatin, cerivastatin, fluvastatin, lovastatin, pitavastatin, pravastatin, rosuvastatin and simvastatin.

20. The consumable film according to claim 1, wherein said water soluble polymer is selected from the group consisting of pullulan, hydrocolloids, β-glucan, maltodextrin, celluloses, including hydroxypropylmethyl cellulose, hydroxyethyl cellulose, hydroxypropyl cellulose, carboxymethyl cellulose, methylcellulose, hydroxymethylcellulose, hydroxyethylcellulose, polyvinyl pyrrolidone, polyvinyl alcohol, sodium alginate, polyethylene glycol, natural gums, such as locust bean gum, carageenen gum, xanthan gum, tragacanth gum, guar gum, acacia gum, arabic gum, karaya, ghatti, tamarind gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, and mixtures thereof.

21. The consumable film according to claim 20, wherein said water soluble polymer is pullulan.

22. The consumable film of claim 21, comprising about 40 to about 80 wt % pullulan;
about 0.01 to about 4 wt % thymol; about 0.01 to about 4 wt % methyl salicylate; about 0.01 to about 4 wt % eucalyptol; and about 0.01 to about 15 wt % menthol.

23. The consumable film according to claim 20, further comprising about 0.01 to about 5 wt % of at least one stabilizing agent; about 0.001 to about 0.1 wt % of at least one of at least one coloring agent; about 0.1 to about 8 wt % of water; about 0.1 to about 15 wt % of at least one sweetening agent; about 0.1 to about 15 wt % of at least one flavoring agent; about 0.1 to about 4 wt % of at least one cooling agent; and about 0.1 to about 5 wt % of at least one surfactant.

24. The consumable film according to according to claim 23, wherein said least one stabilizing agent is selected from the group consisting of xanthan gum, locust bean gum and carrageenan, and said at least one sweetening agent is selected from the group consisting of saccharin, aspartame and acesulfame K.

25. The consumable film according to claim 1, wherein said film does not substantially adhere to itself.

26. The consumable film according to claim 1, further comprising water in an amount from about 3 wt % to about 8 wt %.

27. A method for preparing an edible film comprising an active drug, said method comprising: mixing at least one water soluble film former to provide a film-forming mixture; adding an active drug to the film forming mixture; casting the film forming mixture comprising the active drug on a substrate; and drying the cast film to provide said edible film comprising said active drug.

28. A method for delivering an effective amount of active drug to the oral cavity comprising introducing in the oral cavity the consumable film according to claim 1.

29. An edible film comprising an active drug for use in transmucosal delivery of the active drug to a patient, said film comprising:
a binding agent which is dissolvable in the mouth of the patient; and,
a pharmacologically effective dose of an active drug dispersed in the binding agent to form a mixture that is fashioned into a film such that when the film dissolves in the mouth of the patient, the pharmacologically effective dose of the active drug is released.
